(19) United States
(12) Patent Application Publication
(10) Pub. No.: US 2010/0210718 A1
(43) Pub. Date: Aug. 19, 2010

(54) SYNERGISTIC ANTI-OXIDANT TREATMENT FOR LIPOTOXICITY AND OTHER METABOLICALLY RELATED PHENOMENA

(75) Inventors: Oren TIROSH, Nes Ziona (IL); Zecharia Madar, Rehovot (IL); Anna Aronis, Petach Tikvah (IL); Erez Ilan, Kibbutz Netzer Sireni (IL)

Correspondence Address: BROWDY AND NEIMARK, P.L.L.C.;624 NINTH STREET, NW, SUITE 300, WASHINGTON, DC, 20001-5303, US

(73) Assignee: YISSUM RESEARCH DEVELOPMENT COMPANY OF THE HEBREW UNIVERSITY OF JERUSALEM LTD., Jerusalem (IL)

(21) Appl. No.: 12/633,999

(22) Filed: Dec. 9, 2009

Related U.S. Application Data
(63) Continuation-in-part of application No. 11/783,609, filed on Apr. 10, 2007, now abandoned.
(60) Provisional application No. 60/790,543, filed on Apr. 10, 2006.

(51) Int. Cl. A61K 31/375; A61P 3/10; A61P 9/00; A61P 9/12; A61P 29/00; C12N 5/07

(52) U.S. Cl. 514/474; 435/375

(57) ABSTRACT

A method for ameliorating, reducing, and/or preventing lipotoxicity-induced metabolic dysfunction in at least one cell is disclosed, said method comprising a step of contacting said at least one cell with a therapeutically effective amount of a synergistic anti-lipotoxicity cocktail, said cocktail comprising N-acetylcysteine, ascorbic acid, and resveratrol. Also disclosed is a method for treating at least one lipotoxicity-related metabolic disorder chosen from the group comprising (a) metabolic syndrome; (b) non-alcoholic steatohepatitis; (c) obesity; and (d) coronary heart disease, said method comprising the step of administering to a patient a therapeutically effective amount of an anti-lipotoxicity cocktail, said cocktail comprising N-acetylcysteine, ascorbic acid, and resveratrol. Finally, a composition for treating the effects of lipotoxicity, containing a synergistic combination of N-acetylcysteine, ascorbic acid, and resveratrol is disclosed.

### CROSS-REFERENCE TO RELATED APPLICATIONS

This application is a Continuation-in-Part of U.S. patent application Ser. No. 11/783,609, entitled “Means and Method for Treating Lipotoxicity and Other Metabolically Related Phenomena,” filed on Apr. 10, 2007, which claims priority from U.S. Provisional application 60/790,543, filed on Apr. 10, 2006.

### FIELD OF THE INVENTION

The present invention generally relates to methods and compositions for treating lipotoxicity and other metabolically related phenomena. In particular, it relates to methods and compositions that increase the viability of cells exposed to lipids via administration of synergistic combinations of anti-oxidants.

### BACKGROUND OF THE INVENTION

Obesity is generally recognized as a serious health concern in the developed world. It is associated with a number of other health problems such as Type 2 diabetes, heart disease, inflammation, and hypertension. Overloading of white adipose tissue beyond its storage capacity leads to lipid disorders in non-adipose tissues, namely skeletal and cardiac muscles, pancreas, and liver, effects that are often mediated through increased non-esterified fatty acid fluxes. This in turn leads to a tissue-specific disordered insulin response and increased lipid deposition and lipotoxicity, coupled to abnormal plasma metabolic or lipoprotein profiles or both.

Obesity is only one example of a condition associated with problems of fat metabolism. Metabolic syndrome is another important example. Obesity may play a role in the causation of metabolic syndrome. It has been observed, however, that many of the complications related to obesity are more closely related to the distribution of fat within the body rather than the overall level of obesity. The ultimate cause of metabolic syndrome, however, remains unclear. Predisposing factors include genetic factors, central adiposity, diabetes mellitus, high-fat diets, aging, medications, physical inactivity, polycystic ovary syndrome, and low birth weight.

Type 2 diabetes is another condition associated with abnormalities of fat metabolism. It is generally accepted that both beta-cell dysfunction and reduced insulin sensitivity play a role in the pathogenesis of diabetes, but the exact role of each of these factors remains poorly understood. Central visceral adiposity is found in a majority of individuals suffering from type 2 diabetes. This observation has led to the hypothesis that adipose tissue plays a critical role in the pathogenesis of type 2 diabetes. Recent research has provided evidence that ectopic fat storage syndrome (deposition of triglycerides in muscle, liver, and pancreatic cells) and disruption of normal endocrine function of adipose tissue increase the risk of type 2 diabetes.

The link between obesity and an increased risk for coronary heart disease is well-known. Oxidized low-density lipoprotein (oxLDL) has been shown to play an important role in the pathogenesis of atherosclerosis. In middle-aged people, obesity and dyslipidemia are the strongest predictors of elevated oxLDL levels. The association between dyslipidemia and oxidation of LDL has been demonstrated in pre-diabetic individuals. Furthermore, metabolic syndrome is associated with high risk for atherosclerotic disease as well.

Recently, attention has been focused on excessive accumulation of triglycerides within the liver as a part of metabolic syndrome. The decline in β-cell function observed in individuals suffering from type 2 diabetes has been attributed on the one hand to glucose toxicity (i.e., hyperglycemia due to diabetes leads to damage of β-cells) and lipotoxicity on the other (i.e., β-cell dysfunction is due to hyperlipidemia). Oxidative stress, in which elevated glucose concentrations increase levels of reactive oxygen species in β-cells, is a result of type 2 diabetes. The extent to which this oxidative stress is due solely to hyperglycemia relative to the extent to which it is due to additional complications of hyperlipidemia remains poorly understood.

Fat accumulation in the liver is associated with several features of insulin resistance even in normal weight and moderately overweight (i.e. not obese) subjects. It remains unclear, however, the extent to which hepatic steatosis is a cause rather than effect of metabolic syndrome.

In general, there is continuous cycling and redistribution of non-oxidized fatty acids between different organs. The amount of triacylglycerol (TAG) in a normal liver is not fixed but can readily be increased by nutritional, metabolic, and endocrine interactions involving partitioning between TAG and free fatty acid (FFA) and TAG/FFA metabolism. Several lines of evidence indicated that hepatic TAG accumulation is also a causative factor in development of hepatic insulin resistance. The liver appears to participate both actively and passively in the development of metabolic syndrome. The TAG content of hepatocytes is regulated by the integrated activities of cellular molecules that facilitate hepatic TAG uptake, fatty acid synthesis, and esterification on the one hand (“input”) and hepatic fatty acid oxidation and TG export on the other (“output”). Steatosis occurs when the “input” exceeds the capacity for “output.” The liver acts in concert with other organs in the orchestration of inter-organ FFA/TAG partitioning.

Another phenomenon associated with insulin resistance is endothelial dysfunction. The endothelium is a dynamic autocrine/paracrine organ that regulates vascular tone and the interaction of the wall of a blood vessel with circulating substances and blood cells. The endothelium produces vasodilators and vasoconstrictors that are in balance under normal physiological conditions. A major vasodilator is nitric oxide (NO), which has multiple vascular-protective actions. In contrast, vasoconstrictors such as angiotensin II, promote vascular damage and inflammation. Andothelial dysfunction is an early step in the atherogenic process. It has recently been found that insulin resistance in the absence of overt type 2 diabetes or metabolic syndrome results in endothelial dysfunction in peripheral and coronary vasculature, and that endothelial dysfunction itself could contribute to insulin resistance. Thus, treatment strategies that attenuate cardiovascular disease may also attenuate insulin resistance progression. Until the ultimate mechanisms of endothelial dysfunction are better understood, early recognition and treatment of risk factors associated with insulin resistance or cardiovascular disease are critical in the prevention of atherosclerosis.

Although they differ in details of presentation and of effects, all of the above conditions share in common that they are associated with dysfunctions of fat metabolism at the cellular level, in particular, lipotoxicity-related phenomena in which cell function is compromised (frequently to the point of cell death). It is thus clear that a method of treatment of lipotoxicity-related phenomena in which the treatment acts to block the effects of lipotoxicity at the cellular level would fulfill a long-felt need.

The following literature citations relating to lipotoxicity-related phenomena as discussed above are hereby incorporated by reference: (1) Sonnenberg et al. Obes. Res. 12, 180 (2004). (2) Bray, G. A. et al. J. Am. Dietet. Assn. 104, 86 (2004). (3) Kopp, W. Metabolism 52 (2003), 840. (4) Brook, R. D. et al. J. Am. Coll. Nutr. 22, 290 (2003). (5) Onat, A. et al. Atherosclerosis 168, 81 (2003). (6) Garber, A. J. Am. Fam. Physician 62, 2633 (2000). (7) Sakkinen, P. A. et al. Am. J. Epidemiol. 152, 897 (2000). (8) Tracy, R. P. Int. J. Clin. Pract. Suppl. 134, 10 (2003). (9) Timar, O. Can. J. Cardiol. 16, 779 (2000). (10) Wajchenberg, B. L. Endocr. Rev. 21, 697 (2000). (11) Bouchardet, C. et al. Endocr. Rev. 14, 72 (1993). (12) Lebovitz, H. E. Int. J. Clin. Pract. Suppl. 134, 18 (2003). (13) Yao, M. et al. Int. J. Obes. Relat. Metab. Disord. 27, 920 (2003). (14) Zhu, S.; Wang, Z. et al. Am. J. Clin. Nutr. 78, 228 (2003). (15) Sathyaprakash, R. et al. Curr. Diab. Rep. 2, 416 (2002). (16) Axen, K. V. et al. J. Nutr. 133, 2244 (2003). (17) Bray, G. A.; Ryan, D. H. Endocrine 13, 167 (2000). (18) Everson, S. A. et al. Diabet. Care 21, 1637 (1998). (19) Korytkowski, M. T. et al. J. Clin. Endocrinol. Metab. 80, 3327 (1995). (20) Gustat, J. et al. J. Clin. Epidemiol. 55, 997 (2002). (21) Liu, S. et al. Curr. Opin. Lipidol. 12, 395 (2001). (22) Dahlgren, E. et al. Fertil. Steril. 61, 455 (1994). (23) Catalano, P. M. et al. J. Nutr. 133 Suppl. 2, 1674S (2003). (24) Schubert, C. Nature Med. 10, 322 (2004). (25) Grundy, S. M. JAMA 290, 3000 (2003). (26) Diehl, A. M. Am. J. Physiol. Gastrointest. Liver Physiol. 282, G1-G5 (2002). (27) Scheen, A. J. Acta Clin Belg. 58, 335 (2003). (28) Faraj, M. et al., Biochem. Cell Biol. 82, 170 (2004). (29) Pittas, A. G. et al., Nutr. Clin. Care 6, 79 (2003). (30) Clapham, J. C. et al., Curr. Drug Targets 5, 309 (2004). (31) Shirai, K. Curr. Med. Res. Opin. 20, 295 (2004). (32) Corella, D., and Ordovas, J. M. Curr. Atheroscler. Rep. 6, 186 (2004). (33) Semenkovich, C. F. Trends Cardiovasc Med. 14, 72 (2004). (34) Pankow, J. S.; Jacobs, D. R., Jr; Steinberger, J.; Moran, A.; Sinaiko, A. R. Diabetes Care 27, 775 (2004). (35) Fisher, M. Heart 90, 336 (2004). (36) Holvoet, P. Diabetes 53, 1068 (2004). (37) Ferrannini, E. et al. N. Engl. J. Med. 317, 350 (1987). (38) Reaven, G. M. Diabet. Care 27, 1011 (2004). (39) (Ridker, P. M. Circulation 107, 363 (2003). (40) Frohlich, M. et al. Eur. Heart J. 24, 1365 (2003). (41) den Boer, M. et al. Arterioscler. Thromb. Vasc. Biol. 24, 644 (2004). (42) Bugianesi, E. et al. Dig. Liver Dis. 36, 165 (2004). (43) Robertson, R. P. et al. Diabetes 53, S119 (2004). (44) Fernandez-Checa, J. C. Ann. Hepatol. 2, 69 (2003). (44) Hsueh, W. A.; Quiñones, M. J. Amer. J. Cardiol. 92, 10 (2003). (45) Yokoyama, M. Curr. Opin. Pharmacol. 4, 110 (2004). (46) Higashi Y, Yoshizumi, M. Pharmacol Ther. 102, 87 (2004). (47) Kawano, H.; Ogawa, H. Curr. Drug Targets Cardiovasc. Haematol. Disord. 4, 23 (2004). (47) Ilan, E.; Tirosh, O.; Madar, Z. J. Nutr. 135, 2090 (2005). (48) Aronis, A.; Madar, Z.; Tirosh, O. Free Radical Biol. Med. 38, 1221 (2005).

### SUMMARY OF THE INVENTION

It is thus an object of the present invention to disclose a method of treating lipotoxicity-induced metabolic dysfunction in at least one cell, said method comprising a step of contacting said at least one cell with a therapeutically effective amount of a synergistic anti-lipotoxicity cocktail, said cocktail comprising N-acetylcysteine, ascorbic acid, and resveratrol.

It is a further object of this invention to disclose such a method, wherein said cocktail comprises N-acetylcysteine, ascorbic acid, and resveratrol in a concentration ratio of about 1.0:1.0:0.4.

It is a further object of this invention to disclose such a method, wherein said cocktail comprises about 50 μmol L⁻¹ N-acetylcysteine, about 50 μmol L⁻¹ ascorbic acid, and about 20 μmol L⁻¹ resveratrol.

It is a further object of this invention to disclose such a method, wherein said cocktail comprises N-acetylcysteine, ascorbic acid, and resveratrol in a concentration ratio of between about 1.0:1.0:0.02 and about 1.0:1.0:0.4.

It is a further object of this invention to disclose such a method, wherein said cocktail comprises about 50 μmol L⁻¹ N-acetylcysteine, about 50 μmol L⁻¹ ascorbic acid, and between about 1 μmol L⁻¹ and about 20 μmol L⁻¹ resveratrol.

It is a further object of this invention to disclose such a method, wherein said lipotoxicity-induced metabolic dysfunction leads to decreased cell viability and/or increased probability of cell death.

It is a further object of this invention to disclose such a method, wherein said lipotoxicity-induced metabolic dysfunction is chosen from the group consisting of (a) increased production of reactive oxygen species; (b) increased DNA fragmentation; (c) oxidative stress; (d) suppression of caspase activity; (e) suppression of the apoptotic cell death pathway; (f) activation of the necrotic cell death pathway; and (g) any combination of the above.

It is a further object of this invention to disclose such a method, wherein viability of cells exposed to at least one of (a) lipids, (b) free fatty acid, (c) triglycerides, or (d) any combination of the above and treated according to said method is at least 30% greater than the sum of (a) the cell viability of similarly exposed cells that are treated only with the amount of N-acetylcysteine found in said cocktail, (b) the cell viability of similarly exposed cells that are treated only with the amount of ascorbic acid found in said cocktail, and (c) the cell viability of similarly exposed cells that are treated only with the amount of resveratrol found in said cocktail.

It is a further object of this invention to disclose such a method, wherein viability of cells exposed to at least one of (a) lipids, (b) free fatty acid, (c) triglycerides, or (d) any combination of the above and treated according to said method is at least about 3 times greater than the viability of similarly exposed cells that are treated with only one of the components of said cocktail in the amount in which said component is found in said cocktail.

It is a further object of this invention to disclose such a method, wherein said method provides substantially full protection against triglyceride-induced lipotoxicity.

It is a further object of this invention to disclose such a method, wherein said cell is a macrophage.

It is a further object of this invention to disclose a method for treating at least one lipotoxicity-related metabolic disorder chosen from the group comprising (a) metabolic syndrome; (b) non-alcoholic steatohepatitis; (c) obesity; and (d) coronary heart disease, said method comprising the step of introducing into a patient a therapeutically effective amount of an anti-lipotoxicity cocktail, said cocktail comprising N-acetylcysteine, ascorbic acid, and resveratrol.

It is a further object of this invention to disclose such a method, wherein said cocktail comprises N-acetylcysteine, ascorbic acid, and resveratrol in a concentration ratio of about 1.0:1.0:0.4.

It is a further object of this invention to disclose such a method, wherein said cocktail comprises about 50 μmol L⁻¹ N-acetylcysteine, about 50 μmol ^{L-1} ascorbic acid, and about 20 μmol L⁻¹ resveratrol.

It is a further object of this invention to disclose such a method, wherein said cocktail comprises N-acetylcysteine, ascorbic acid, and resveratrol in a concentration ratio of between about 1.0:1.0:0.02 and about 1.0:1.0:0.4.

It is a further object of this invention to disclose such a method, wherein said cocktail comprises about 50 μmol L⁻¹ N-acetylcysteine, about 50 μmol L⁻¹ ascorbic acid, and between about 1 μmol L⁻¹ and about 20 μmol L⁻¹ resveratrol.

It is a further object of this invention to disclose such a method, wherein viability of cells exposed to at least one of (a) lipids, (b) free fatty acid, (c) triglycerides, or (d) any combination of the above is at least 30% greater than the sum of (a) the cell viability of similarly exposed cells that are treated only with the amount of N-acetylcysteine found in said cocktail, (b) the cell viability of similarly exposed cells that are treated only with the amount of ascorbic acid found in said cocktail, and (c) the cell viability of similarly exposed cells that are treated only with the amount of resveratrol found in said cocktail.

It is a further object of this invention to disclose such a method, wherein viability of cells exposed to at least one of (a) lipids, (b) free fatty acid, (c) triglycerides, or (d) any combination of the above and treated according to said method is at least about 3 times greater than the viability of similarly exposed cells that are treated with only one of the components of said cocktail in the amount in which said component is found in said cocktail.

It is a further object of this invention to disclose such a method, wherein said method provides substantially full protection against triglyceride-induced lipotoxicity.

It is a further object of this invention to disclose such a method, wherein said cocktail is introduced into said patient orally.

It is a further object of this invention to disclose such a method, wherein said cocktail is introduced into said patient intravenously.

It is a further object of this invention to disclose a composition for treating at least one condition chosen from (a) lipotoxicity-induced metabolic dysfunction in at least one cell; (b) metabolic syndrome; (c) non-alcoholic steatohepatitis; (d) lipotoxic effects of obesity; and (e) coronary heart disease; said composition comprising predetermined quantities of N-acetylcysteine, ascorbic acid, and resveratrol, wherein the effect of treatment with said composition shows a synergistic effect relative to treatment using any one of its components alone.

It is a further object of this invention to disclose such a composition, wherein said composition comprises N-acetylcysteine, ascorbic acid, and resveratrol in a ratio of about 1.0:1.0:0.4 by volume.

It is a further object of this invention to disclose such a composition, wherein said composition comprises N-acetylcysteine, ascorbic acid, and resveratrol in concentrations of about 50 μmol L⁻¹, 50 μmol L⁻¹, and 20 μmol L⁻¹, respectively.

It is a further object of this invention to disclose such a composition, wherein said composition comprises N-acetylcysteine, ascorbic acid, and resveratrol in a concentration ratio of between about 1.0:1.0:0.02 and about 1.0:1.0:0.4.

It is a further object of this invention to disclose such a composition, wherein said composition comprises about 50 μmol L⁻¹ N-acetylcysteine, about 50 μmol L⁻¹ ascorbic acid, and between about 1 μmol L⁻¹ and about 20 μmol L⁻¹ resveratrol.

It is a further object of this invention to disclose such a composition, adapted for oral administration.

It is a further object of this invention to disclose such a composition, adapted for intravenous administration.

### BRIEF DESCRIPTION OF THE FIGURES

In order to understand the invention and to see how it may be implemented in practice, a preferred embodiment will now be described, by way of non-limiting example only, with reference to the accompanying figures in which:

FIG. 1 illustrates cellular uptake of fatty acids and dose-dependent lipotoxicity following exposure of J774.2 macrophage cells to a lipid emulsion (LE);

FIG. 2 illustrates levels of reactive oxygen species (ROS) and DNA fragmentation following exposure of J774.2 macrophage cells to an LE;

FIG. 3 illustrates changes in intracellular concentrations of ROS, superoxide, and reduced glutathione following exposure of J774.2 macrophage cells to an LE;

FIG. 4 illustrates cell viability and caspase-3 activity in J774.2 macrophage cells exposed to an LE;

FIG. 5 illustrates the effect of exposure to a proapoptotic protein-synthesis inhibitor on ROS concentration and caspase-3 activity in J774.2 macrophage cells exposed to an LE;

FIG. 6 illustrates the synergistic effect of one embodiment of the current invention as a treatment for lipotoxicity-related metabolic dysfunction; and

FIG. 7 illustrates the synergistic effects of additional embodiments of the current invention as treatments for lipotoxicity-related metabolic dysfunction.

### DETAILED DESCRIPTION OF THE PREFERRED EMBODIMENTS

In the following description, various aspects of the invention will be described. For the purposes of explanation, specific details are set forth in order to provide a thorough understanding of the invention. It will be apparent to one skilled in the art that there are other embodiments of the invention that differ in details without affecting the essential nature thereof. Therefore the invention is not limited by that which is illustrated in the figures and described in the specification, but only as indicated in the accompanying claims, with the proper scope determined only by the broadest interpretation of said claims.

As used herein, the term “lipotoxicity-related phenomenon” refers to any phenomenon associated with a metabolic disorder selected from a group consisting of Metabolic Syndrome, lipotoxicity, coronary heart disease, non-alcoholic steatohepatitis, any cellular dysfunction due to cellular interaction with triacylglycerol, free fatty acid, or lipids, or any combination thereof.

As used herein, the term “treating” a condition refers to any means for curing, ameliorating, lessening the severity of, or otherwise reducing at least some of the negative effects of the condition. The term also includes preventative treatment, that is, treatment that prevents, lessens the likelihood of contracting, lessens the effects of, or lessens the severity of the condition upon exposure to a causative factor of the condition.

The following abbreviations are used herein:

“AA” refers to ascorbic acid.

“CH” refers to cycloheximide.

“FACS” refers to flow cytometry.

“FFA” refers to free fatty acid(s).

“GSH” refers to reduced glutathione.

“LE” refers to lipid emulsion.

“NAC” refers to N-acetyl cysteine.

“PI” refers to propidinium iodide.

“ROS” refers to reactive oxygen species.

“TAG” and “TG” refer to triacylglycerol (triglyceride).

The present invention discloses a novel method for treating lipotoxicity-induced cellular metabolic dysfunction by treating cells with a synergistic combination of selected anti-oxidants. In a preferred embodiment of the invention, the method comprises contacting said cells with a therapeutic amount of an anti-liptoxicity cocktail, said anti-lipotoxicity cocktail comprising N-acetylcysteine (NAC), ascorbic acid (AA), and resveratrol. In a more preferred embodiment of the invention, the anti-lipotoxicity cocktail comprises NAC, AA, and resveratrol in a volume ratio of about 1.0:1.0:0.4 (NAC:AA:resveratrol). In a most preferred embodiment of the invention, the anti-lipotoxicity cocktail comprises about 50 μmol L⁻¹ NAC, about 50 μmol L⁻¹ AA, and about 20 μmol L⁻¹ resveratrol. In alternative embodiments of the invention, the anti-lipotoxicity cocktail comprises a smaller proportion of resveratrol such that the overall ratio is between about 1.0:1.0:0.02 and about 1.0:1.0:0.4. In additional alternative embodiments of the invention, the anti-lipotoxicity cocktail comprises about 50 μmol L⁻¹ NAC, about 50 μmol L⁻¹ AA, and between about 1 μmol L⁻¹ and about 20 μmol L⁻¹ resveratrol. The synergistic effect of the anti-lipotoxicity cocktail relative to the effects of its individual components is demonstrated in the examples given below.

The method for contacting the cell or cells with said anti-lipotoxicity cocktail can be any method for doing so known in the art. In a most preferred embodiment, the cocktail is introduced into the environment surrounding the cell(s) to be treated, and the components then diffuse into the cell. While in a preferred embodiment, the cocktail is prepared before the treatment and introduced into the environment surrounding the cell(s) as a single composition, in alternative embodiments, the components may be introduced into the environment surrounding the cell(s) in any order.

In one embodiment of the invention, the method is used to treat any type of cell that suffers from metabolic dysfunction due to the effects of lipotoxicity. In a preferred embodiment, the cells treated are macrophages.

In a preferred embodiment of the invention, the method is used to treat cells that have already been affected by lipotoxicity. In other embodiments of the invention, the method is used to protect healthy cells from the effects of exposure to TAG, lipids, and/or fatty acids.

It is also within the scope of this invention to disclose a method for treating at least one lipotoxicity-related metabolic disorder chosen from the group comprising (a) metabolic syndrome; (b) non-alcoholic steatohepatitis; (c) obesity; and (d) coronary heart disease. In one embodiment, the method comprises the step of introducing into a patient a therapeutically effective amount of an anti-lipotoxicity cocktail, said cocktail comprising N-acetylcysteine, ascorbic acid, and resveratrol. In a more preferred embodiment of the invention, the anti-lipotoxicity cocktail comprises NAC, AA, and resveratrol in a volume ratio of about 1.0:1.0:0.4 (NAC:AA:resveratrol). In a most preferred embodiment of the invention, the anti-lipotoxicity cocktail comprises about 50 μmol L⁻¹ NAC, about 50 μmol L⁻¹ AA, and about 20 μmol L⁻¹ resveratrol. In alternative embodiments of the invention, the anti-lipotoxicity cocktail comprises a smaller proportion of resveratrol such that the overall ratio is between about 1.0:1.0:0.02 and about 1.0:1.0:0.4. In additional alternative embodiments of the invention, the anti-lipotoxicity cocktail comprises about 50 μmol L⁻¹ NAC, about 50 μmol L⁻¹ AA, and between about 1 μmol L⁻¹ and about 20 μmol L⁻¹ resveratrol.

The anti-lipotoxicity cocktail can be introduced into the patient by any method known in the art that is convenient for the patient and the caregiver. In one preferred embodiment, the anti-lipotoxicity cocktail is introduced orally, while in another preferred embodiment, it is introduced intravenously.

It is also within the scope of the invention to disclose a composition for treating the following conditions: (a) lipotoxicity-induced metabolic dysfunction in at least one cell; (b) metabolic syndrome; (c) non-alcoholic steatohepatitis; (d) obesity; and (e) coronary heart disease. As discussed in detail above, all of these conditions are related to or are manifestations of lipotoxicity at the cellular level. As shown in the examples given below, this composition is effective in treating these effects at the cellular level. In one embodiment of the invention, the composition comprises predetermined quantities of N-acetylcysteine, ascorbic acid, and resveratrol. In a more preferred embodiment of the invention, the composition comprises NAC, AA, and resveratrol in a volume ratio of about 1.0:1.0:0.4 (NAC:AA:resveratrol). In a most preferred embodiment of the invention, the composition comprises about 50 μmol L⁻¹ NAC, about 50 μmol L⁻¹ AA, and about 20 μmol L⁻¹ resveratrol. In alternative embodiments of the invention, the composition comprises a smaller proportion of resveratrol such that the overall ratio is between about 1.0:1.0:0.02 and about 1.0:1.0:0.4. In additional alternative embodiments of the invention, the composition comprises about 50 μmol L⁻¹ NAC, about 50 μmol L⁻¹ AA, and between about 1 μmol L⁻¹ and about 20 mol L⁻¹ resveratrol.

In alternative embodiments of the invention, the composition herein disclosed is adapted for oral administration (e.g. by preparation as a liquid, suspension, or emulsion). In some embodiments of the invention, the composition is adapted for oral administration by placing a therapeutically effective dose within a delivery device (e.g. a capsule) adapted for oral administration of therapeutic materials. Such capsules are well-known in the art. In other alternative embodiments of the invention, the composition is adapted for intravenous administration according to any of the methods well-known in the art. In a preferred embodiment, the composition is prepared prior to its administration in combination. In other embodiments, the individual components are administered separately in any order, the time between administrations of individual components being less than the time needed by the body to metabolize the previously administered component(s), i.e., in embodiments in which different components are administered at different times, the entire course of administration will take place over a short enough period of time that all three components are simultaneously present in the body of the patient in order to ensure that the synergistic effect of the composition will occur.

### EXAMPLE 1

The following example illustrates how the method and composition herein disclosed may be used in practice. First, the effects of lipotoxicity on the viability of J774.2 macrophages are demonstrated. The therapeutic effect of the method and composition herein disclosed, and evidence for a synergistic effect relative to the effect of any one of the components of the composition, are then demonstrated. The example is intended to be illustrative of the use of the method and composition, and not to limit in any way the scope of the invention as described and claimed.

A. Experimental Methods

Experimental methods used in the measurement of the effects of exposure of Murine J774.2 macrophages to a lipid emulsion (LE), and for the demonstration of the therapeutic effect of the method and composition disclosed herein, are given in detail in Aronis, A.; Madar, Z.; Tirosh, O. Free Rad. Biol. Med. 38, 1221 (2005). A brief summary is given here.

1. Cell Culture.

Murine J774.2 macrophages were cultured in RPMI medium enriched with 10% fetal calf serum, 1% glutamine, and 1% penicillin-streptomycin. Cells were maintained in an incubator with temperature (378 C) and CO₂ (5%) control. Prior to experimental procedures, macrophages were seeded on 6-well plates at a concentration of 50,000 cells/ml.

2. LE Treatment.

Soybean oil based LE was added to the cell culture at a concentration of 0.1% (w/v) TAG (1 mg lipids/ml). The physiological range of TAG in plasma is up to 1.5 mg/ml. The cells were incubated with the LE for 12, 24, or 48 h, and then washed twice with phosphate-buffered saline (PBS), and intracellular ROS and cell viability were measured. Other agents, such as antioxidants or cycloheximide (CH), were added as specified below.

3. Determination of Cellular Fatty Acid Profile.

The cellular concentrations of fatty acids were measured using gas chromatography (GC). Quantification of the results was based on the peak area of a known amount of C₁₇ fatty acid added as an internal standard.

4. Cell Viability.

Cell membrane integrity was measured by staining the cells with 2 μg/ml propidium iodide (PI) and then using flow cytometry (FACS) to measure fluorescence emission at 575 nm (488 nm excitation). Data were collected from 10,000 cells.

5. DNA Integrity.

Cells exposed to LE were centrifuged (600 g, 5 min). The pellet was resuspended in 1% (w/v) paraformaldehyde, incubated for 30 min, and centrifuged. The resultant pellet was resuspended in a solution containing 50 μg/ml PI, 0.1% (w/v) sodium citrate, and 0.1% (v/v) Triton X-100. The permeabilized cells were kept in the dark at 48° C. for 2 h. DNA integrity was analyzed by FACS, excitation at 488 nm and emission at 575 nm. Data were collected from 10,000 cells.

6. ROS Measurements.

Intracellular ROS were dectected using an H₂DCF-DA fluorescent probe. Fluorescence emission (488 nm excitation) at 530 nm was measured using FACS. Superoxide was measured by measurement of the absorbance at 520 nm following treatment with nitroblue tetrazolium.

7. Measurement of Glutathione.

GSH was measured by high-pressure liquid chromatography with an electrochemical detector.

8. Statistical Analysis.

Statistical analyses of the data were performed by ANOVA. In the following discussion, differences are considered to be statistically significant at a probability level P<0.05. In the figures, statistically significant differences are indicated by different letters; in a given graph, two results marked with the same letter do not differ significantly.

B. Demonstration of the Lipotoxic Effect of LE Exposure on J774.2 Macrophages

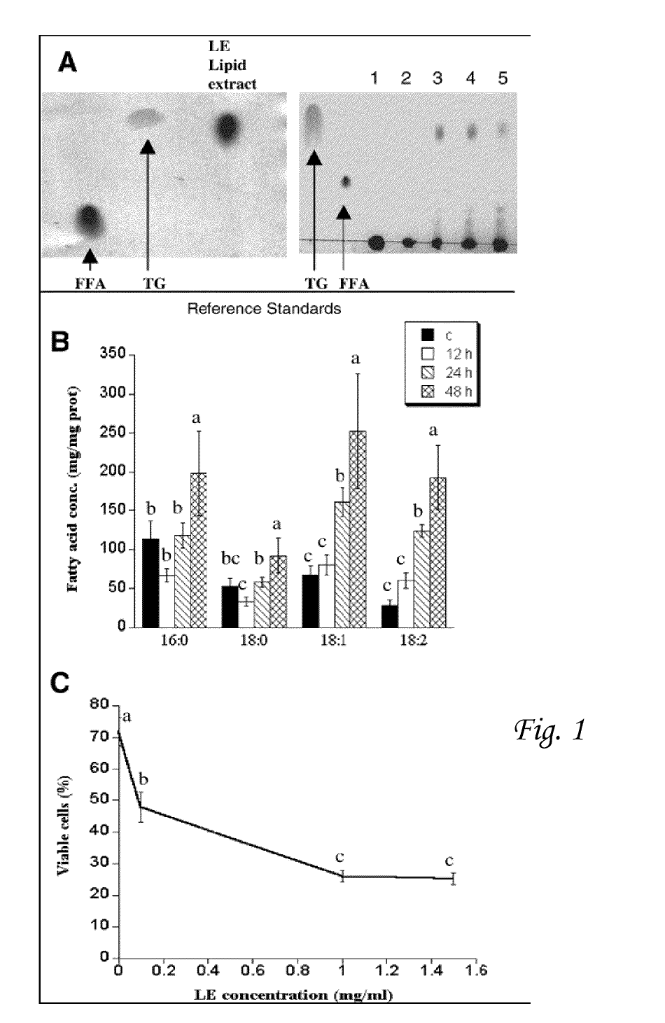

Reference is now made to FIG. 1, which shows results of exposure of J774.2 macrophages to LE. As shown by the TLC results presented in the left panel of FIG. 1a, the LE was essentially free of FFA. TLC results presented in the right-hand panel of FIG. 1a show that the cell culture medium remained essentially free of FFA after exposure of cells in culture to 1 mg lipid/ml TAG for 12 h, 24 h, and 48 h (chromatograms 3, 4, and 5 respectively; chromatograms 1 and 2 are control runs for cells not exposed to LE). FIG. 1b shows cellular concentrations of four different fatty acids following exposure to LE. The increase in cellular levels of 18:1 and 18:2 fatty acids correlates well with the composition of the LE, indicating that the increase in cellular fatty acid concentration was due to incorporation of the LE into the cells. FIG. 1c shows cellular viability as a function of LE concentration, and shows a clear increase in cell death with increasing LE concentration, demonstrating the lipotoxic effect of TAG on macrophage cells.

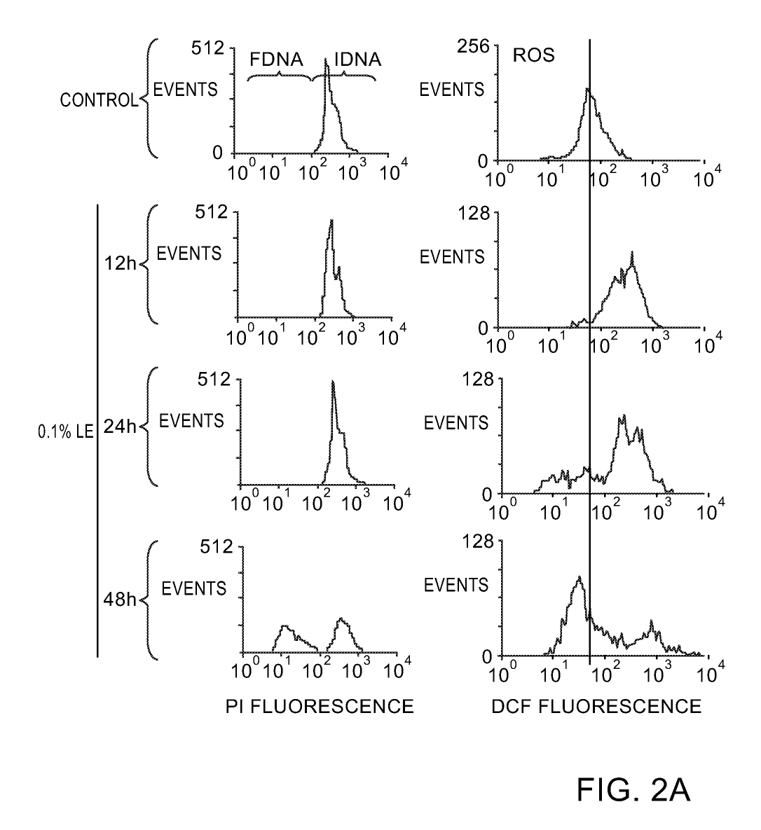

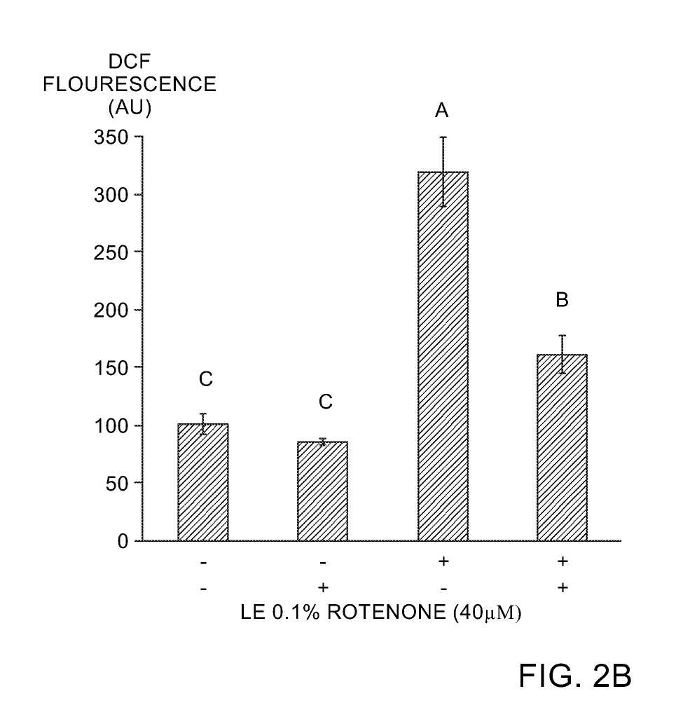

Reference is now made to FIG. 2, which shows the effect of TAG on cellular redox status. The series of graphs on the left side of FIG. 2a represent successive measurements of DNA fragmentation in cells exposed to an LE containing 0.1% TAG for 12 h, 24 h, and 48 h, relative to a control sample that was not exposed to LE. The results shown in these graphs indicate 50% DNA fragmentation after 48 h exposure to LE. The series of graphs on the right side of FIG. 2a show ROS concentrations after 12 h, 24 h, and 48 h of exposure to the same LE, relative to a control sample not exposed to LE. Within 24 h, two populations (low TOS and high TOS) could be identified. After 48 h, the cells with low levels of ROS became dominant. FIG. 2b shows results for cells exposed to the LE and treated with rotenone (a mitochondrial complex 1 inhibitor) 5 min before the ROS measurements were made. The two bars on the left show results for the control sample, while those on the right show results for cells exposed to the LE. These data demonstrate that the ROS were produced by an endogenous intracellular process rather than being delivered from the LE (e.g. in the form of oxidized lipids). Furthermore, it is clear from these results that changes in ROS levels are an early signal of lipotoxicity, occurring before the degradation of the cellular DNA.

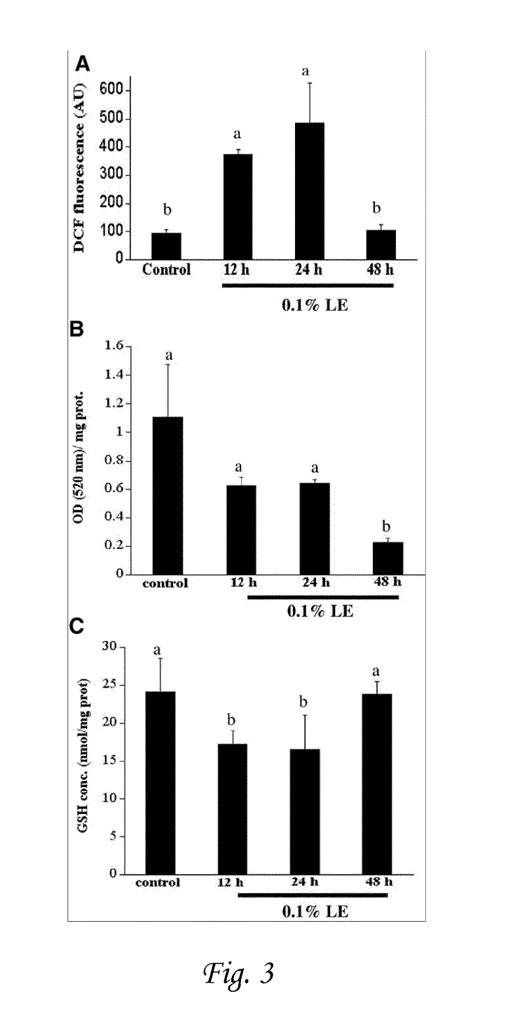

Reference is now made to FIG. 3, which shows results of measurements of ROS (FIG. 3a), superoxide (FIG. 3b), and reduced glutathione (GSH) (FIG. 3c) following 12 h, 24 h, and 48 h exposure to the 0.1% LE. A significant decrease in superoxide levels is seen after 48 h of exposure. Changes in the cellular antioxidant status occurred, reflecting oxidative stress. The measurements of the GSH levels show consumption of this antioxidant in the presence of a high level of ROS.

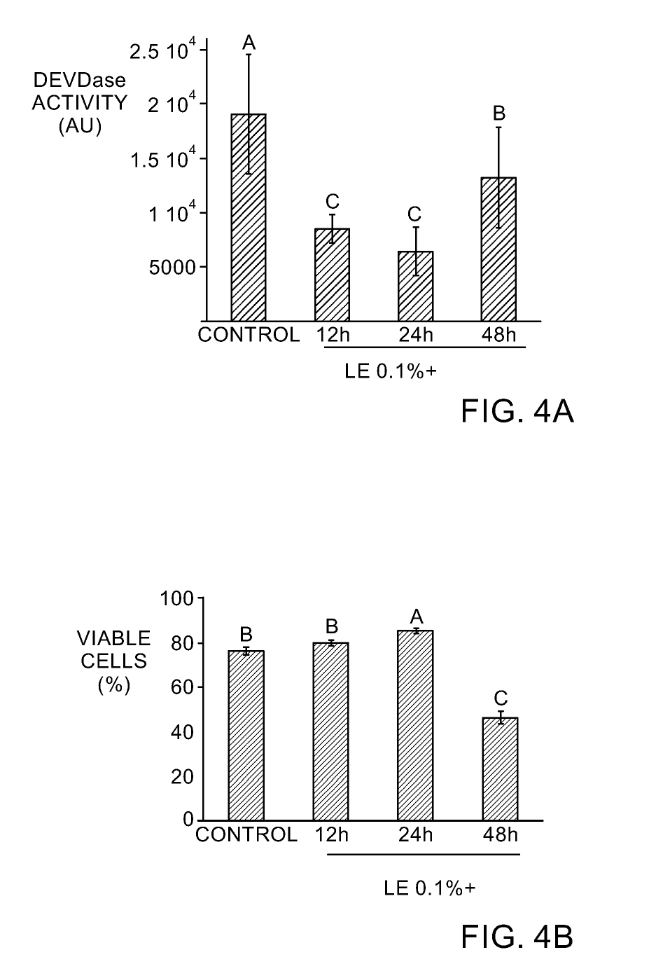

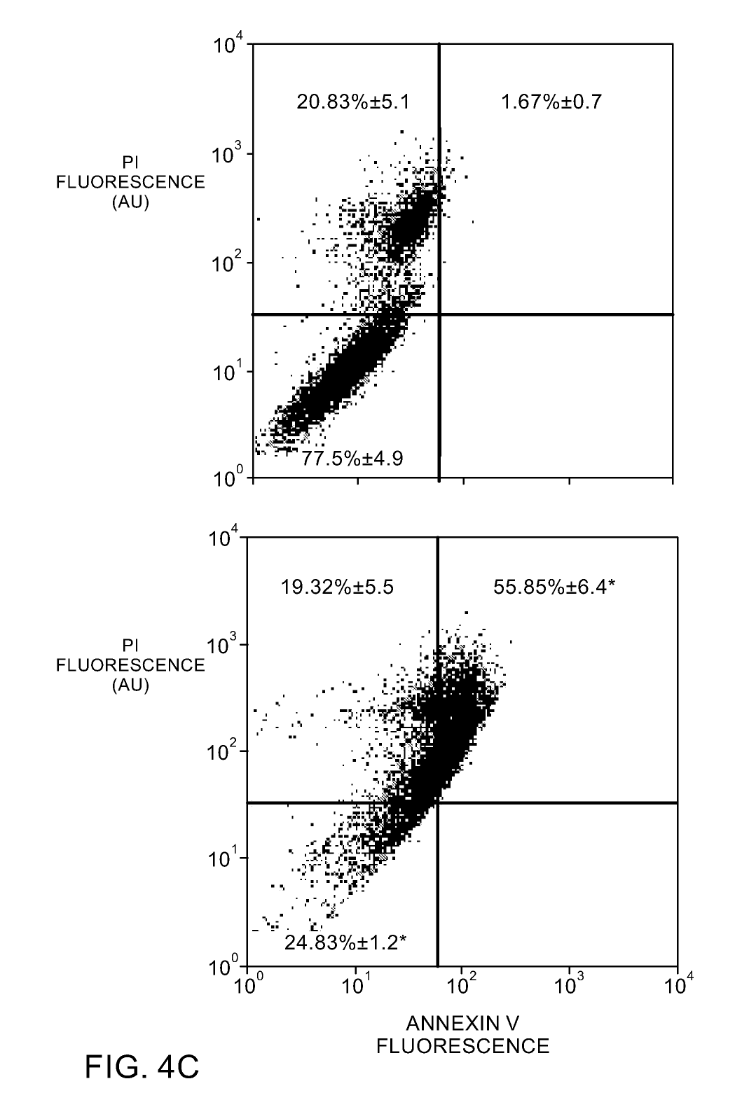

Reference is now made to FIG. 4, which shows measurements of cell viability and caspase-3 activity following exposure to the LE. FIG. 4a shows that treatment with LE did not activate caspase-3 activity, and in fact, basal caspase-3 activity was suppressed by exposure to the LE. After 24 h of exposure to the LE, there was a small but significant increase in the proportion of viable cells relative to the control population. These data indicate that for the first 24 h of exposure, the apoptotic pathway is suppressed in the macrophages, most likely via a high level of ROS, which are known to suppress caspase activity. Longer (48 h) exposure resulted in death of 50% of the cells, as shown in FIG. 4b. In order to examine the relative activation of apoptotic and necrotic cell death pathways, the macrophages were stained with propidinium iodide (PI) and Annexin V. FIG. 4c shows PI fluorescence as a function of Annexin fluorescence. The results shown in the figure indicate activation of the necrotic cell death pathway and necrotic cell death.

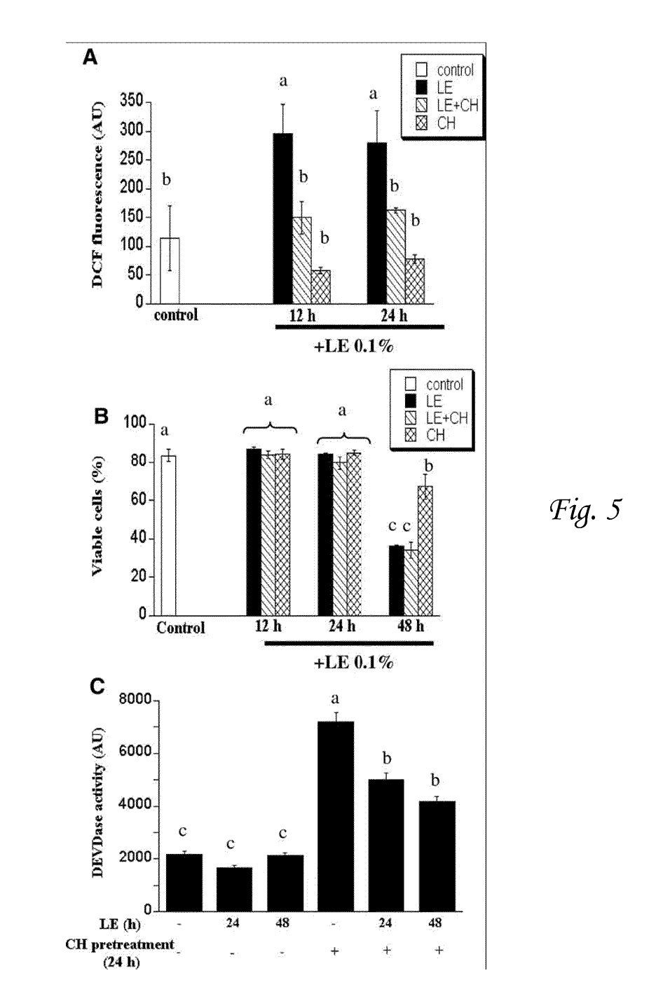

Reference is now made to FIG. 5, which shows results for cells exposed to LE and treated with CH, a proapoptotic protein-synthesis inhibitor. CH is known to activate cellular signaling, resulting in caspase activation and apoptosis. FIG. 5a shows results for cells pretreated with CH. CH treatment alone decreases ROS production in the cells, allowing caspase-3 activity in the higher reducing environment. FIG. 5b shows results of measurements of viable cells following treatment with CH, LE, or both. These results demonstrate that TAG treatment via exposure to the LE following treatment with CH led to cell death within 48 h, indicating that protein-synthesis inhibition does not prevent the cell-death effect of TAG. As shown in FIG. 5c, which shows caspase-3 activity in cells exposed to LE with or without CH treatment, TAG treatment following CH treatment significantly elevates cellular ROS levels and partially suppresses caspase-3 activation. These results indicate that a higher oxidation state in lipotoxicity suppresses caspase-3 activity and intrinsic apoptosis capacity, leading to necrotic cell death.

These results show that J774.2 macrophages are sensitive to TAG-induced lipotoxicity; that TAG causes an elevation in ROS production leading to caspase system inhibition resulting in necrotic cell death; and that the source of the ROS is complex 1 of the mitochondrial electron-transfer chain. Thus, not only are the macrophages themselves an example of a cellular system that is susceptible to metabolic dysfunction as a result of lipotoxicity arising from cellular interactions with lipids absorbed from the environment, they provide a good model at the cellular level for the lipotoxicity-related phenomena and metabolic disorders associated with lipotoxicity in humans.

C. Effect of the Present Invention on Lipotoxicity-induced Cell Death

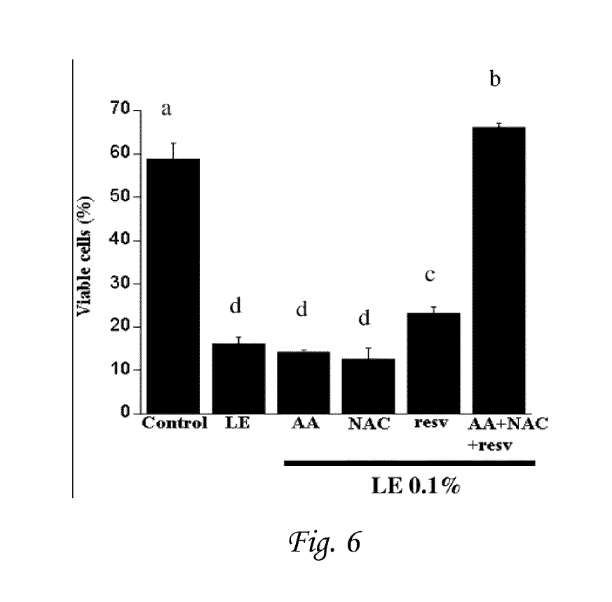

Reference is now made to FIG. 6, which shows the effects on cell viability of macrophages exposed to an LE following treatment with the invention herein disclosed. For a control group of macrophage cells (unexposed to the LE and kept in culture for 48 h), cell viability was approximately 60%. As can be seen, 48 h after exposure to the LE, cell viability dropped below 20%. Neither the presence of 50 μmol L⁻¹ AA or of 50 μmol L⁻¹ NAC during the time that the cells were exposed to the LE had any statistically significant effect on cell viability. When the cells were exposed to the LE in the presence of 20 μmol L⁻¹ resveratrol, there was a slight improvement in the cell viability, to approximately 20%. When the cells were exposed to LE in the presence of all three, however, the cell viability was over 65%, i.e., better than in the control population.

This result is surprising for at least two reasons. First, the general assumption in development of treatments for lipotoxicity and the syndromes associated with it is that lipid-soluble antioxidants would be most effective treatments. That a combination of water-soluble antioxidants should show such a dramatic effect is unexpected.

Even more surprising is the synergistic effect of the combination disclosed in the present invention. If the action of each of the three components of the “anti-lipotoxicity cocktail” (AA, NAC, and resveratrol) were independent of the others, and assuming that the cell viability upon exposure to LE in the presence of AA and NAC represents their maximum level of activity, then we would expect that the cell viability upon exposure to LE in the presence of all three would be no more than the sum of the individual viabilities, approximately 50%. In fact, the cell viability was approximately 65%, some 30% greater than would be expected from a linear combination of cell viabilities. If, on the other hand, the lack of a statistically significant improvement in cell viability when AA and NAC were used alone indicates that they are ineffective when used alone, then there would be no reason to expect that the simultaneous presence of AA, NAC, and resveratrol would have any greater effect than resveratrol alone. As the results shown in the figure demonstrate, the actual cell viability achieved when AA, NAC, and resveratrol are all present when the cells are exposed to LE is approximately 3 times that of the cell viability achieved from the presence of resveratrol alone. The improvement in cell viability when the “cocktail” is used relative to that when resveratrol alone is used is a factor of approximately 9; resveratrol alone increases the cell viability by approximately 5%, while the “cocktail” increases it by approximately 45%. Thus, no matter what definition of the expected effect of a linear combination of the three components of the “cocktail” is used, it is clear that the effect in practice is significantly greater, and that the method and composition disclosed in the present invention show an unexpected synergistic effect in the treatment of lipotoxicity-related metabolic dysfunction. In the present example, the method and composition herein disclosed provide substantially full protection against the effects of lipotoxicity.

### EXAMPLE 2

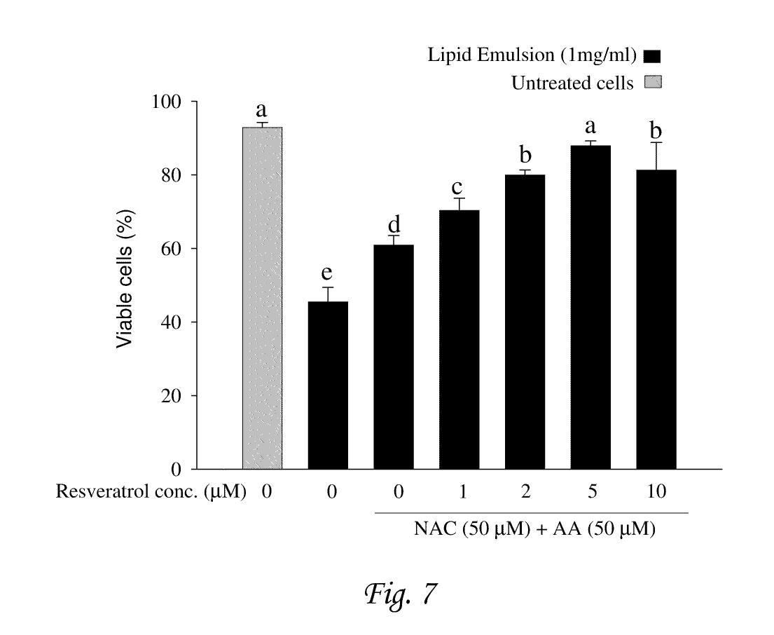

Reference is now made to FIG. 7, which illustrates the effects of additional embodiments of the present invention. In this series of experiments, the macrophage cells were exposed to LE in the presence of 50 μmol L⁻¹ NAC and 50 μmol L⁻¹ AA with varying amounts of resveratrol. These results show that the aforementioned synergistic effect is achieved even with a resveratrol concentration of 1 μmol L⁻¹.

We claim:

1. A method of treating lipotoxicity-induced metabolic dysfunction in at least one cell, said method comprising a step of contacting said at least one cell with a therapeutically effective amount of a synergistic anti-lipotoxicity cocktail, said cocktail comprising N-acetylcysteine, ascorbic acid, and resveratrol.

2. The method of claim 1, wherein said at least one cell is present in the body of a subject.

3. The method of claim 1, wherein said cocktail comprises N-acetylcysteine, ascorbic acid, and resveratrol in a concentration ratio of about 1.0:1.0:0.4.

4. The method of claim 1, wherein said cocktail comprises about 50 μmol L⁻¹ N-acetylcysteine, about 50 μmol L⁻¹ ascorbic acid, and about 20 μmol L⁻¹ resveratrol.

5. The method of claim 1, wherein said cocktail comprises N-acetylcysteine, ascorbic acid, and resveratrol in a concentration ratio of between about 1.0:1.0:0.02 and about 1.0:1.0:0.4.

6. The method of claim 1, wherein said cocktail comprises about 50 μmol L⁻¹ N-acetylcysteine, about 50 μmol L⁻¹ ascorbic acid, and between about 1 μmol L⁻¹ and about 20 μmol L⁻¹ resveratrol.

7. The method of claim 1, wherein said lipotoxicity-induced metabolic dysfunction leads to decreased cell viability and/or increased probability of cell death.

8. The method of claim 1, wherein said lipotoxicity-induced metabolic dysfunction is chosen from the group consisting of (a) increased production of reactive oxygen species; (b) increased DNA fragmentation; (c) oxidative stress; (d) suppression of caspase activity; (e) suppression of the apoptotic cell death pathway; (f) activation of the necrotic cell death pathway; and (g) any combination of the above.

9. The method of claim 1, wherein viability of cells exposed to at least one of (a) lipids, (b) free fatty acid, (c) triglycerides, or (d) any combination of the above and treated according to said method is at least 30% greater than the sum of (a) the cell viability of similarly exposed cells that are treated only with the amount of N-acetylcysteine found in said cocktail, (b) the cell viability of similarly exposed cells that are treated only with the amount of ascorbic acid found in said cocktail, and (c) the cell viability of similarly exposed cells that are treated only with the amount of resveratrol found in said cocktail.

10. The method of claim 1, wherein viability of cells exposed to at least one of (a) lipids, (b) free fatty acid, (c) triglycerides, or (d) any combination of the above and treated according to said method is at least about 3 times greater than the viability of similarly exposed cells that are treated with only one of the components of said cocktail in the amount in which said component is found in said cocktail.

11. The method of claim 1, wherein said method provides substantially full protection against triglyceride-induced lipotoxicity.

12. The method of claim 1, wherein said cell is a macrophage.

13. A method for treating at least one lipotoxicity-related metabolic disorder chosen from the group comprising (a) metabolic syndrome; (b) non-alcoholic steatohepatitis; (c) obesity; and (d) coronary heart disease, said method comprising the step of administering to a patient a therapeutically effective amount of an anti-lipotoxicity cocktail, said cocktail comprising N-acetylcysteine, ascorbic acid, and resveratrol.

14. The method of claim 13, wherein said cocktail comprises N-acetylcysteine, ascorbic acid, and resveratrol in a concentration ratio of about 1.0:1.0:0.4.

15. The method of claim 13, wherein said cocktail comprises about 50 μmol L⁻¹ N-acetylcysteine, about 50 μmol L⁻¹ ascorbic acid, and about 20 μmol L⁻¹ resveratrol.

16. The method of claim 13, wherein said cocktail comprises N-acetylcysteine, ascorbic acid, and resveratrol in a concentration ratio of between about 1.0:1.0:0.02 and about 1.0:1.0:0.4.

17. The method of claim 13, wherein said cocktail comprises about 50 μmol L⁻¹ N-acetylcysteine, about 50 μmol L⁻¹ ascorbic acid, and between about 1 μmol L⁻¹ and about 20 μmol L⁻¹ resveratrol.

18. The method of claim 13, wherein viability of cells exposed to at least one of (a) lipids, (b) free fatty acid, (c) triglycerides, or (d) any combination of the above is at least 30% greater than the sum of (a) the cell viability of similarly exposed cells that are treated only with the amount of N-acetylcysteine found in said cocktail, (b) the cell viability of similarly exposed cells that are treated only with the amount of ascorbic acid found in said cocktail, and (c) the cell viability of similarly exposed cells that are treated only with the amount of resveratrol found in said cocktail.

19. The method of claim 13, wherein viability of cells exposed to at least one of (a) lipids, (b) free fatty acid, (c) triglycerides, or (d) any combination of the above and treated according to said method is at least about 3 times greater than the viability of similarly exposed cells that are treated with only one of the components of said cocktail in the amount in which said component is found in said cocktail.

20. The method of claim 13, wherein said method provides substantially full protection against triglyceride-induced lipotoxicity.

21. The method of claim 13, wherein said cocktail is introduced into said patient orally.

22. The method of claim 13, wherein said cocktail is introduced into said patient intravenously.

23. A composition for treating at least one lipotoxicity-related condition chosen from the group consisting of (a) lipotoxicity-induced metabolic dysfunction in at least one cell; (b) metabolic syndrome; (c) non-alcoholic steatohepatitis; (d) lipotoxic effects of obesity; and (e) coronary heart disease, said composition comprising predetermined quantities of N-acetylcysteine, ascorbic acid, and resveratrol, wherein the effect of treatment with said composition shows a synergistic effect relative to treatment using any one of its components alone.

24. The composition of claim 23, wherein said composition comprises N-acetylcysteine, ascorbic acid, and resveratrol in a ratio of about 5:5:2 by volume.

25. The composition of claim 23, wherein said composition comprises N-acetylcysteine, ascorbic acid, and resveratrol in concentrations of about 50 μmol L⁻¹, 50 μmol L⁻¹, and 20 μmol L⁻¹, respectively.

26. The composition of claim 23, wherein said composition comprises N-acetylcysteine, ascorbic acid, and resveratrol in a concentration ratio of between about 1.0:1.0:0.02 and about 1.0:1.0:0.4.

27. The composition of claim 23, wherein said composition comprises about 50 μmol L⁻¹ N-acetylcysteine, about 50 μmol L⁻¹ ascorbic acid, and between about 1 μmol L⁻¹ and about 20 ∥mol L⁻¹ resveratrol.

28. The composition of claim 23, adapted for oral administration.

29. The composition of claim 23, adapted for intravenous administration.
